(19) United States
(12) Patent Application Publication
(10) Pub. No.: US 2020/0390370 A1
(43) Pub. Date: Dec. 17, 2020

(54) SYSTEM, APPARATUS AND METHOD FOR ACTIVITY CLASSIFICATION

(71) Applicant: MINDMAZE HOLDING SA, Lausanne (CH)

(72) Inventors: Tej TADI, Lausanne (CH); Farzin DADASHI, Lausanne (CH); Karim KANOUN, Lausanne (CH); Benoît MARIANI, Lausanne (CH)

(21) Appl. No.: 16/772,569

(22) PCT Filed: Dec. 12, 2018
(86) PCT No.: PCT/IB2018/059933

§ 371 Date: Jun. 12, 2020

Related U.S. Application Data
(60) Provisional application No. 62/597,646, filed on Dec. 12, 2017.

(51) Int. Cl. A61B 5/11; A61B 5/00

(52) U.S. Cl. CPC A61B 5/1118; A61B 5/743; A61B 5/6802; A61B 5/1112

(57) ABSTRACT

A system, method and apparatus that is capable of automatically detecting and classifying various physical activities of a user. This enables such activities to be analyzed, for example according to the complexity of the activity and the amount of time spent in each activity. A barcode may be calculated, according to the various activities of the user, the amount of time spent in each activity and optionally also the complexity of each such activity.

### FIELD OF THE INVENTION

The present invention, in at least some embodiments, relates to a system, method and apparatus for activity classification, and in particular to such a system, method and apparatus which can automatically detect and differentiate between various physical activities of the user.

### BACKGROUND OF THE INVENTION

Activity trackers, such as the Fitbit device or sports watches, track various physical activities of the user. However they are limited in their ability to provide a comprehensive picture of the user's activities, as these devices must be manually set by the user to detect each such activity.

Other solutions have been proposed to measure such properties as gait characteristics, but these also suffer from drawbacks. For example, JP4971808B2 describes a system that uses video cameras to measure the movement of individual body parts through image processing. The determination of the movement of individual body parts does not provide an analysis of the overall movement of the body.

US Patent Application No. 20160074272 requires sensors to be placed on the waist and legs of the user in specific locations, which is clearly disadvantageous.

### BRIEF SUMMARY OF THE INVENTION

The present invention, in at least some embodiments, provides a system, method and apparatus that is capable of automatically detecting and classifying various physical activities of a user. This enables such activities to be analyzed, for example according to the complexity of the activity and the amount of time spent in each activity. A barcode may be calculated, according to the various activities of the user, the amount of time spent in each activity and optionally also the complexity of each such activity.

The apparatus may comprise a wearable or may be implemented through a cellular telephone of the user. The apparatus comprises an IMU and software for analyzing the activities of the user. The apparatus is preferably in communication with a server, for example to store information in a database or to receive information regarding user activity classification.

According to at least some embodiments, there is provided an apparatus for automatically detecting and classifying physical activities of a user, implemented to be worn, held by or attached to the user, comprising a computational device, an IMU (Inertial Measurement Unit) for collecting angular velocity and linear acceleration data about the user, and software for analyzing the activities of the user; wherein said computational device is configured to perform a predefined set of basic operations in response to receiving a corresponding basic instruction selected from a predefined native instruction set of codes of said software, said instruction set comprising:

a first set of machine codes selected from the native instruction set for receiving said IMU data,

a second set of machine codes selected from the native instruction set for preprocessing said IMU data and

a third set of machine codes selected from the native instruction set for determining an activity of the user according to said IMU data; wherein each of the first, second and third sets of machine code is stored in the memory.

Optionally the apparatus is implemented as a mobile electronic device. Optionally said mobile electronic device comprises a cellular telephone or a smart phone. Optionally the apparatus is implemented as a wearable. Optionally the IMU is contained in a wearable, worn by the user, the computational device comprises a mobile electronic device and analysis is performed by said mobile electronic device upon receiving said IMU data from said wearable.

Optionally said computational device comprises a display and wherein a classified activity of the user is displayed on said display. Optionally said software is capable of determining a length of time each activity of the user is performed, an intensity with which the activity is performed, or both, and to display said length of time, said intensity, or both, on said display.

Optionally the apparatus further comprises a GPS (global positioning system) device, for providing user location information; wherein said software analyzes said user location information as an input for determining a speed of the user in performing an activity. Optionally said software is capable of combining previously determined GPS data and IMU data when current GPS data is not available, to determine a speed of the user from current IMU data.

Optionally the apparatus further comprises a data communication module for communicating said IMU data, said user activity information, or both, with an external device; and for receiving further analysis of said IMU data, said user activity information, or both, from said external device. Optionally said external device comprises a server.

According to some embodiments, there is provided a system, comprising the apparatus as described herein, optionally in any embodiment or combination thereof, said apparatus further comprising a data communication module for communicating said IMU data, said user activity information, or both, and further comprising a server in communication with said data communication module of said apparatus, said server further comprising a database and a classifier for classifying an activity of the user according to said IMU data, said user activity information, or both.

According to some embodiments, there is provided a method for analyzing physical activities of a user with the apparatus or system as described herein, optionally in any embodiment or combination thereof, comprising automatically detecting a category of physical activity of a user according to signals from the IMU.

Optionally the method further comprises automatically determining an amount of time spent in each activity. Optionally the method further comprises automatically determining a complexity of each activity. Optionally said automatically determining said complexity comprises determining an entropy of said activity, wherein a higher entropy indicates a higher complexity.

Optionally the method further comprises calculating a barcode of the physical activities of the user, wherein said calculating comprises determining a physical activity type, duration, intensity and sequence of user activities. Optionally the method further comprises receiving IMU signals from the IMU; conditioning said IMU signals to form IMU data; extracting a plurality of biomechanical parameters from said IMU data; and classifying the category of physical activity of the user according to said biomechanical parameters.

Optionally said conditioning said IMU signals to form IMU data comprises performing a dynamic calibration so axes of the IMU are virtually aligned to functional movement axes of a movement of the user. Optionally said performing said dynamic calibration comprises performing an optimization that minimizes the difference between virtually-rotated-IMU signals and the functional movement axes of body segments of a body of the user.

Optionally said extracting said biomechanical parameters comprises determining duration of movement, interpretation of intensity, calculation of velocity and IMU orientation in 3D space. Optionally said determining interpretation of intensity comprises determining an intensity of performance of an activity. Optionally said extracting said biomechanical parameters comprises determining a geometric shape of the IMU signal at each physical activity cycle and determining time series coefficients for the IMU signal. Optionally said classifying comprises performing a basic activity classification and an advanced activity classification, wherein said basic activity classification comprises determining an initial activity classification, and said advanced activity classification comprises determining the category of physical activity according to said initial activity classification and a temporal sequence of activity in terms of previous user activities. Optionally said basic activity classification further comprises applying dynamic time warping to the IMU data. Optionally said basic activity classification further comprises classifying the activity according to IMU signal amplitude, auto-regressive coefficients that describe each cycle of IMU data and signal form features extracted from the dynamic time warping.

Optionally the method further comprises obtaining concurrent GPS data and IMU data; correlating said GPS data and said IMU data to determine a speed of a user activity; obtaining further IMU data without GPS data; and determining said speed according to a previous correlation of said GPS data and said IMU data.

U.S. Pat. No. 9,043,251 describes a system for determining unit behaviors, based on an accelerometer, GPS and the like. Based upon these unit behaviors, the system attempts to classify user activity as a whole. However, the unit behaviors are not determined by considered the activity of the user as a whole, but only in atomized fashion, which may result in an imprecise or incorrect activity classification.

U.S. Pat. No. 9,125,015 also atomizes information provided from a plurality of sensors in a mobile device, by attempting to classify user activities according to signals received from each sensor separately. Separate classification based on each signal is likely to result in information loss and hence inaccurate classification.

U.S. Pat. No. 9,460,398 relates to acceleration data for classification of a user's activity. However, the method is limited to Fast Fourier Transform analysis, such that it is limited to time sequence analysis only.

U.S. Pat. No. 9,769,166 trains an engine based on accelerometer data from a user, to be able to classify that user's activities and to identify the user. However the system also requires a muscle sensor, such as an electrical based electromyography (EMG) sensor, to be worn by the user.

U.S. Pat. No. 9,999,395 relates to a two step process for activity classification, in which the location of a mobile device containing sensors is first determined, such as for example in the “pocket” or “hands” of the user. Next, accelerometer data is used to classify a user activity, although details are not provided as to how the data is used for such classification.

U.S. patent Ser. No. 10/127,565 relates to a classification method for determining a user activity in order to select advertising. However, the method requires the user to initially manually determine or correct the classified activity, so that the system can learn from the user's specific data.

Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

Although the present invention is described with regard to a “computing device”, a “computer”, a “computational device” or “mobile device”, it should be noted that optionally any device featuring a data processor and the ability to execute one or more instructions may be described as a computer, including but not limited to any type of personal computer (PC), a server, a distributed server, a virtual server, a cloud computing platform, a cellular telephone, an IP telephone, a smartphone, or a PDA (personal digital assistant). Any two or more of such devices in communication with each other may optionally comprise a “network” or a “computer network”.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the drawings:

FIGS. 1A-1C show three non-limiting, exemplary systems according to at least some embodiments of the present invention, for classifying a physical activity of a user;

FIG. 2 shows a non-limiting, exemplary method for classifying an activity of a user;

FIG. 3 shows another exemplary, non-limiting method for classification of user activities, with the addition of determination of user speed through GPS;

FIG. 4 shows another non-limiting exemplary method for using GPS data to calculate speed; and

FIG. 5 shows a non-limiting, exemplary screenshot of an app for use with the user device and/or wearable device described herein.

### DESCRIPTION OF AT LEAST SOME EMBODIMENTS

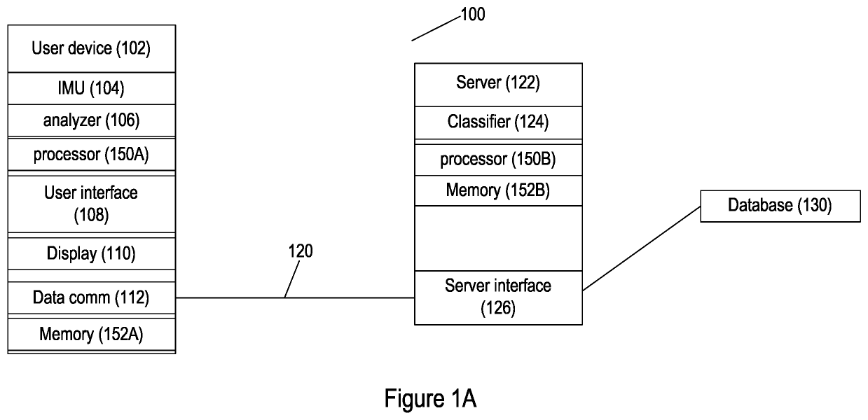

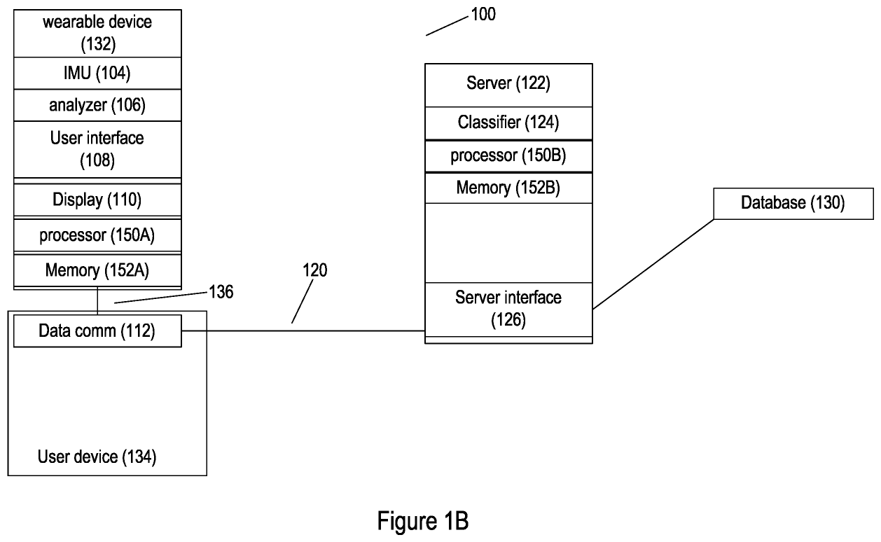

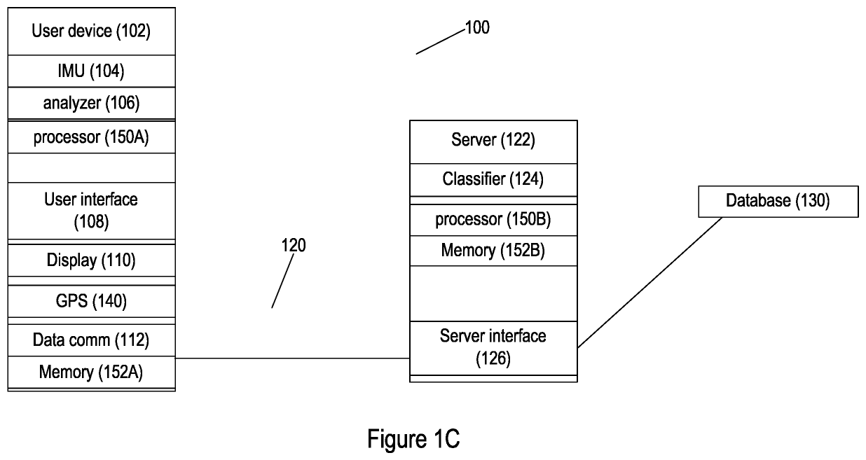

Turning now to the drawings, FIGS. 1A-1C show three non-limiting, exemplary systems according to at least some embodiments of the present invention, for classifying a physical activity of a user.

As shown, FIG. 1A shows a system 100, featuring a user device 102. In this non-limiting example, user device 102 is assumed to be a mobile communications device, such as a cellular telephone for example or any other suitable computational device. User device 102 is preferably able to perform most, if not all, of the analysis functions independently. Such functions are preferably performed according to sets of machine instructions, stored in a memory 152A and processed by a processor 150A. However, user device 102 is preferably in communication with a remote server 122 through a computer network 120 as shown, for additional services, and optionally to access and/or share additional data. Computer network 120 may optionally comprise the Internet for example. The user is assumed to be holding, wearing or otherwise to be attached to user device 102, such that movements of the user are reflected in movements of user device 102.

User device 102 features an IMU (inertial measurement unit) 104 for collecting angular velocity and linear acceleration data, in regard to movements of user device 102, thereby collecting such data about movements of the user. IMU 104 is preferably selected for a suitable sensitivity and range, according to the functions described in greater detail below. The operations of user device 102 and the processing of signals from IMU 104, as well as the analysis thereof, is supported by processor 150A according to instructions stored in memory 152A.

As used herein, a processor generally refers to a device or combination of devices having circuitry used for implementing the communication and/or logic functions of a particular system. For example, a processor may include a digital signal processor device, a microprocessor device, and various analog-to-digital converters, digital-to-analog converters, and other support circuits and/or combinations of the foregoing. Control and signal processing functions of the system are allocated between these processing devices according to their respective capabilities. The processor may further include functionality to operate one or more software programs based on computer-executable program code thereof, which may be stored in a memory. As the phrase is used herein, the processor may be “configured to” perform a certain function in a variety of ways, including, for example, by having one or more general-purpose circuits perform the function by executing particular computer-executable program code embodied in computer-readable medium, and/or by having one or more application-specific circuits perform the function.

User device 102, which may also be described as a computational device, is preferably configured to perform a predefined set of basic operations in response to receiving a corresponding basic instruction selected from a predefined native instruction set of codes. The instruction set comprises at least a first set of machine codes selected from the native instruction set for receiving the IMU data, a second set of machine codes selected from the native instruction set for preprocessing the IMU data and a third set of machine codes selected from the native instruction set for determining an activity of the user according to the IMU data, as described in greater detail below with regard to the activity of an analyzer 106. Each of the first, second and third sets of machine code is stored in the memory 152A, and is executed by processor 150A. For example, by “receiving” the IMU data, it is meant that processor 150A executes instructions that enable the IMU data to be received from IMU 104 for processing.

An analyzer 106 receives such data from IMU 104 and then classifies an activity of the user according to such data. As described in greater detail below, such activity classification preferably features selecting a category of such activity, such as for example sitting, standing, walking or running. More preferably, such activity classification includes determining a complexity of the user's activities overall, which may optionally be expressed in various ways. As a non-limiting example, the user's activities may optionally be transformed into a barcode, which displays both the complexity and length of time of the user's activities.

The results of such classification, optionally including the barcode, may be displayed on a display 110, which may be integrally formed with or attached to user device 102. User device 102 also preferably features a user interface 108, which is displayed on display 110. Preferably, the results of the classification, as well as other visual information, is displayed through user interface 108 by display 110. User interface 108 also preferably accepts user commands, as display 110 is preferably a touchscreen. For example, the user may optionally select which data is to be displayed and for which time period.

As used herein, a “user interface” 108 generally includes a plurality of interface devices and/or software that allow a user to input commands and data to direct the processing device to execute instructions. For example, the user interface may include a graphical user interface (GUI) or an interface to input computer-executable instructions that direct the processor to carry out specific functions. The user interface employs certain input and output devices to input data received from a user or output data to a user. These input and output devices may include a display, mouse, keyboard, button, touchpad, touch screen, microphone, speaker, LED, light, joystick, switch, buzzer, bell, and/or other customer input/output device for communicating with one or more users.

Alternatively user commands may be input through a separate user input device (not shown), which may, for example, comprise a keyboard, mouse, other pointing device, touchscreen, and the like.

User device 102 also preferably features a data communication module 112, for communicating with a server interface 126 of server 122 through computer network 120 as previously described. The operations of server 122 are supported by a processor 150B, according to instructions stored in a memory 152B.

Data from IMU 104 and/or analysis results from analyzer 106 may be shared with server 122 through such communication. Such data and/or analysis results then may optionally be stored in a database 130. Optionally such data and/or analysis results are shared through a social sharing platform, including but not limited to Facebook, Twitter, Snapchat, Whatsapp and the like.

Server 122 may also optionally feature a classifier 124, for performing part or all of the activity classification process. Although the methods as described herein are described as being performed on a single device, optionally the methods may be performed partially on a plurality of devices, in a distributed manner.

FIG. 1B shows an alternative configuration of the system of FIG. 1A, in which various functions performed by the user device of FIG. 1A are instead performed by a wearable device 132. Wearable device 132 may optionally comprise a wristwatch, wristband or other wearable device that is worn by the user. Wearable device 132 comprises IMU 104, analyzer 106, user interface 108 and display 110, with the same or similar functions as described with regard to the user device of FIG. 1A.

A user device 134 is optionally in communication with wearable device 132, for example through a wired or wireless connection 136. Such communication may for example enable the user to view the data on user device 134, through a display (not shown) or to perform various functions with regard to wearable device 132. In this non-limiting example, user device 134 may be a mobile device, such as a cellular telephone for example.

Optionally user device 134 supports communication between wearable device 132 and server 122 as previously described, through data communication module 112. Alternatively, wearable device 132 communicates directly with server 122 (not shown).

FIG. 1C shows another variation on the system of FIG. 1A. In this non-limiting example, user device 102 further comprises a GPS (global positioning system) device 140. GPS device 140 enables user location to be more precisely determined, for example for determining user speed, as described in greater detail below. GPS device 140 may also optionally be combined with the system of FIG. 1B, as part of the wearable device (not shown).

In this configuration, user device 102 is preferably configured as a self-learning running tracker. A self-learning tracker uses both geolocalization and motion data to compute with the highest precision gait and running speed. The speed calculation model is updated during outdoor activities or in the presence of a connected treadmill. If GPS (or treadmill as reference values for speed) is not available, the self-learning tracker, provided through analyzer 106 for example, instead provides personally tailored tracker of running (walking) speed according to previously determined outdoor measurements or connected treadmill measurements. The algorithmic intelligence is based on advanced machine learning techniques to improve indoor tracking by fusing IMU data with GPS data during outdoor measurements.

Within either the user device or the wearable device, optionally the following components are included as a non-limiting implementation example:
Input 3D Acc @32 Hz only, for example
Embedded C library with minimal footprint (12 kb allocated memory for execution on Nucleo F4, for example)

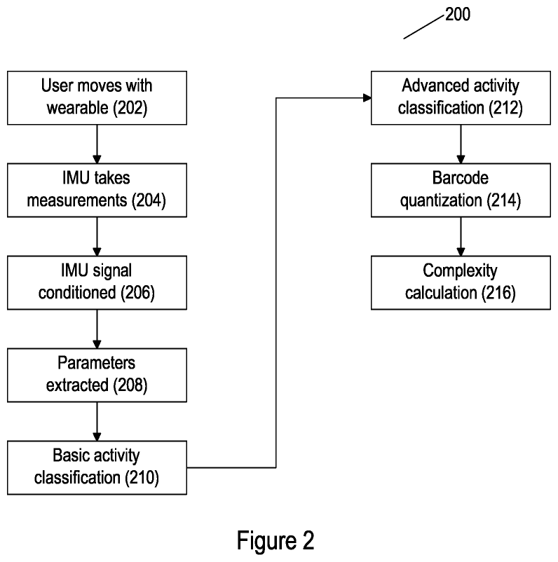

FIG. 2 shows a non-limiting, exemplary method for classifying an activity of a user. The method may optionally be performed with any of the systems of FIGS. 1A-1C. As shown, a method 200 begins with the user moving with a wearable device in stage 202. Although reference is made to a “wearable device”, optionally the user device of FIGS. 1A and 1C could also be used.

The IMU takes measurements as the user moves in stage 204. In stage 206, the IMU signals are conditioned. Such signal conditioning preferably includes performing a dynamic calibration so IMU axes are virtually aligned to the functional movement axes. The calibration is preferably performed as an optimization that minimizes the difference between virtually-rotated-IMU signals and the functional movement axes of body segments of a body of the user. Such a calibration means that the analyzer is able to determine the activity parameters without requiring specific direction of attachment of IMU to the user body.

In stage 208, various parameters are extracted, with regard to biomechanical parameters. This stage features the application of signal processing methods to extract information about duration of movement, interpretation of intensity, calculation of velocity and IMU orientation in 3D space. Interpretation of intensity is performed for example to automatically categorize the intensity of activity, for example in such categories as sedentary, normal or high intensity activity (training).

Optionally, in this stage, the method is based on extraction of cycle by cycle statistical features. The feature extraction method at this stage is insusceptible to physical activity cycle duration or amplitude and mainly dependent on geometric shape of the IMU signal at each cycle. The IMU signal is preferably represented as a time series which is a concise way of describing the signal for the duration of observation. Therefore, the time series coefficients are used to reconstruct the signal, and indeed, may be all the information required (in a root min squared error sense) to reconstruct the signal. This way the information content of the signal will be compressed into a few coefficients that are used as features for activity classification.

In stage 210, activity classification is performed, or at least a portion of such classification is performed. Once the IMU is aligned to the bodily axis (signal conditioning, from stage 204) and movement generic parameters are extracted, then the activity type can be classified.

Preferably, as shown, activity classification is performed in two stages: stage 210, in which a basic activity classification is performed; and stage 212, in which advanced activity classification is performed. In this implementation, the activity labeling is performed via a hybrid classification at the two stages.

In stage 210, a classification is performed, which provides a label for the type of activity and a confidence interval on the certainty of chosen label. Optionally dynamic time warping is applied to the data, to account for temporal effects. For example, the classification may be performed according to multi-class QDA (quadratic discriminant analysis), a technique which is well known in the art. Other suitable techniques include but are not limited to Hidden Markov Models, linear classifiers, multi layer perceptron, deep neural network, radial basis functions, and support vector machines. The features used for the covariance matrix of the QDA preferably include, but are not limited to, statistical features such as signal amplitude, auto-regressive coefficients that describe each cycle of IMU data (preferably in 6 channels), and signal form features extracted from the dynamic time warping.

In stage 212, expert rules are preferably applied, based on the output of the classifier and temporal sequence of activity in terms of previous activities. As a non-limiting example of an expert rule, if a sitting activity has been detected as the previous activity, but a change in the activity has been determined, then if the current activity is also detected as sitting, the latter classification is preferably rejected and the activity will be classified as the next most probable type in a probabilistic sense. Once a change of activity has been detected, then a new category of activity is preferably determined, as a non-limiting example of an expert rule. According to the application of such rules, the activity type is preferably accepted or modified.

In stage 214, barcode quantization is then performed. By determining physical activity type, duration, intensity and sequence a barcode can be calculated. Each physical activity has continuous intensity range which imposes curse of dimensionality for a later stage of calculating the complexity. In order to reduce the noise as well as complexity computational cost, the physical activity intensity is preferably quantized based on an optimization process.

The barcode optionally includes the following parameters in regard to each of the user's physical activities or groups of activities:

1. Type: lying, sitting, standing, . . . .

2. Duration: sit-stand duration, sedentary vs. active periods

3. Intensity: Acceleration, velocity of movement, cadence

4. Pattern: Temporal sequence of activity types

5. Context: Indoor vs outdoor

Some non-limiting examples of the activity types and exemplary metrics that can be measured are given below:
Activity classes (Metrics shown in italics):
   Resting (duration)
   Sitting (duration)
   Standing still (sway jerkiness)
   . . . random movements (duration)
   Walking (steps, cadence, speed)
   Running (steps, cadence, speed)

Once activity type, duration, intensity and sequence have been determined, the temporal sequence of different physical activities is preferably visualized as a barcode. The structural complexity of this barcode characterizes pain/frailty related physical activity and behavior of individuals. Physical activity quantification based on the above characteristics is also a key tool for user energy expenditure assessment.

In stage 216, complexity calculation is then performed. Entropy measures have been used to estimate the amount of “complexity” in a physiological system. A behavior with a greater degree of dynamical complexity shows higher entropy. Existing complexity metrics are based on a single time scale that limits the scope of interpretation to only that level and does not fully capture the dynamics of the entire system. The barcode complexity can be represented at multiple time scales using multi-scale-entropy (MSE). which allows to determine specific time scales at which pain/movement deficits occurs.

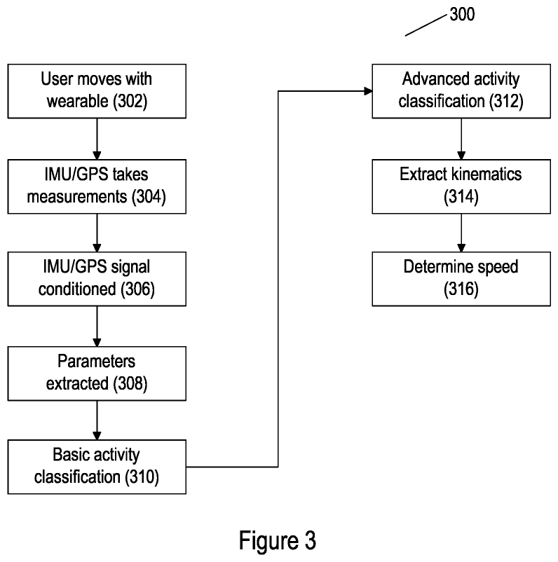

FIG. 3 shows another exemplary, non-limiting method for classification of user activities, with the addition of determination of user speed through GPS. FIG. 3 may optionally be performed with the system of FIG. 1C, for example.

As shown, a method 300 starts with the user moving while wearing a wearable device in stage 302, similar to stage 202 of FIG. 2. In stage 304, both IMU and GPS measurements are collected.

In stage 306, both IMU and GPS signals are preferably conditioned. The IMU signal may optionally be conditioned as previously described. The GPS signals may optionally be conditioned according to any suitable method, as is known in the art. In stage 308, biomechanical parameters are extracted as described with regard to stage 208 of FIG. 2. Stages 310 and 312 are preferably performed similarly as stages 210 and 212 of FIG. 2.

In stage 314, run (walk) kinematics features are preferably extracted, if the activity type is classified as run or walk. Extraction of these kinematics is only possible if the context of the movement is known. The algorithm is applicable based on the data collected from different sites of the body including Wrist, Head, Chest. This block also involves feature whitening and dimension reduction.

In stage 316, a speed estimation is performed, for example using the lolimot speed estimator updater. A Locally Linear Model Tree (LoLiMoT) is a subset of fuzzy algorithms for input space decomposition with local linear least squares optimization. The input space is decomposed in an axis-orthogonal manner yielding hyper-rectangles which accommodate fuzzy membership functions. The standard deviation of membership functions are chosen proportionally to the extension of hyper-rectangle. LoLiMoT provides a flexible framework to model intrinsic nonlinearities between input and output spaces. LoLiMoT was used to find the model that maps Kinematics features to running (walking) speed.

If reference data is available, the LoLiMoT estimator, updates the mathematical speed model of the user. If the user is in the areas where GPS (reference) data is not available or decides to turn of GPS to save battery, then a recently updated model can accurately estimate running (walking) speed based on the last modification of the model. Once the user feels that her/his technique has changed after a few month she/he can recalibrate the model by turning on the GPS for a few minutes during a training session.

Suitable reference data is used to train (update) the speed calculation model. The data can be received from GPS during outdoor running (walking) or via connection to indoor references such as connected treadmills.

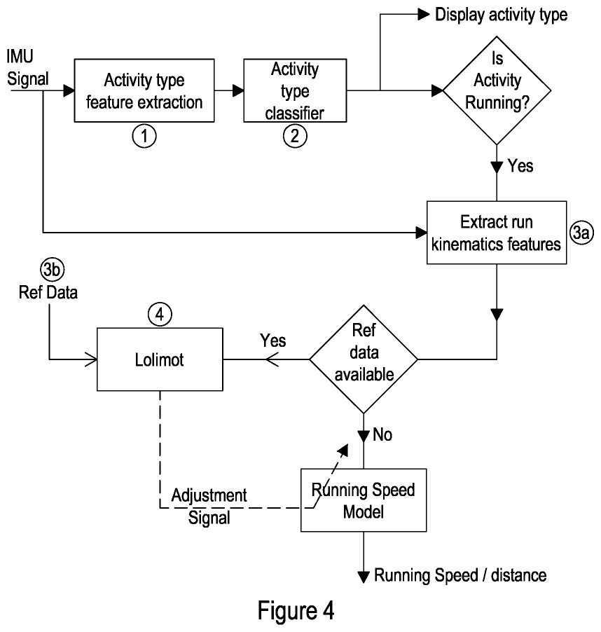

FIG. 4 shows another non-limiting exemplary method for using GPS data to calculate speed. The numbers below correspond to numbers in the drawing.

[1] ACTIVITY TYPE FEATURE EXTRACTION: The block is based on extraction of cycle by cycle statistical features. The feature extraction method at this stage is insusceptible to cycle duration or amplitude and mainly dependent on geometric shape of the IMU signal at each cycle.

[2] ACTIVITY TYPE CLASSIFIER: Is from the family of quadratic discriminant analysis (multi-class). This classifier may be trained after feature extraction using time series coefficients, as described above.

[3a] EXTRACT RUN (WALK) KINEMATICS FEATURES: Beside the features extracted by [1] if the activity type is classified as run (walk) a new set of kinematics features will be extracted at this stage. Extraction of these kinematics is only possible if the context of the movement is known. The algorithm is applicable based on the data collected from different sites of the body including Wrist, Head, Chest. This block also preferably involves feature whitening and dimension reduction. Feature whitening may be performed to provide equal importance to extracted features and reduce numerical issues in classification.

Dimension reduction may be performed to remove unnecessary information, due to high correlation between features. This way the information will be presented in a more compact way to the classifier and computational cost is reduced.

[3b] REFERENCE DATA: This data is used to train (update) the speed calculation model. The data can be received from GPS during outdoor running (walking) or via connecting to indoor references such as connected treadmills. This data is used as previously described.

[4] LOLIMOT SPEED ESTIMATOR UPDATER: Locally Linear Model Tree (LoLiMoT) is a subset of fuzzy algorithms for input space decomposition with local linear least squares optimization. The input space is decomposed in an axis-orthogonal manner yielding hyper-rectangles which accommodate fuzzy membership functions. The standard deviations of membership functions are chosen proportionally to the extension of hyper-rectangle. LoLiMoT provides a flexible framework to model intrinsic nonlinearities between input (3a)-output (3b) spaces. LoLiMoT may be used for example to find the model that maps Kinematics features to running (walking) speed.

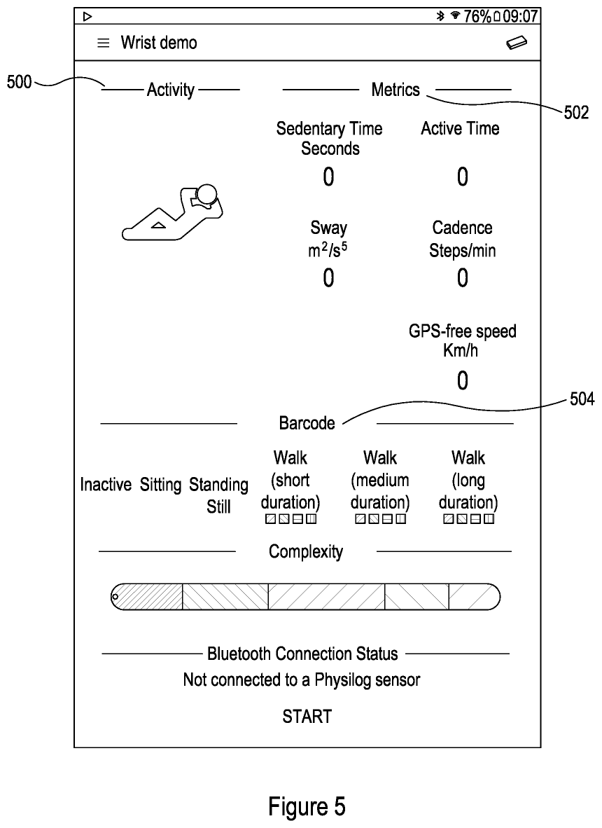

FIG. 5 shows a non-limiting, exemplary screenshot of an app for use with the user device and/or wearable device described herein. As shown in the screenshot, an activity 500 of the user is shown at the top left corner. At the top right corner, various metrics 502 for the current activity and/or other activities are shown. A barcode 504 as described herein is also shown, demonstrating a sequence of activities, the intensity of each and the amount of time spent on each.

Embodiments of the present invention are described above with reference to flowchart illustrations and/or block diagrams of methods, apparatus (systems), and computer program products. It will be understood that each block of the flowchart illustrations and/or block diagrams, and/or combinations of blocks in the flowchart illustrations and/or block diagrams, can be implemented by computer-executable program code portions. These computer-executable program code portions may be provided to a processor of a general purpose computer, special purpose computer, or other programmable data processing apparatus to produce a particular machine, such that the code portions, which execute via the processor of the computer or other programmable data processing apparatus, create mechanisms for implementing the functions/acts specified in the flowchart and/or block diagram block or blocks.

These computer-executable program code portions may also be stored in a computer-readable memory that can direct a computer or other programmable data processing apparatus to function in a particular manner, such that the code portions stored in the computer readable memory produce an article of manufacture including instruction mechanisms which implement the function/act specified in the flowchart and/or block diagram block(s).

The computer-executable program code may also be loaded onto a computer or other programmable data processing apparatus to cause a series of operational steps to be performed on the computer or other programmable apparatus to produce a computer-implemented process such that the code portions which execute on the computer or other programmable apparatus provide steps for implementing the functions/acts specified in the flowchart and/or block diagram block(s). Alternatively, computer program implemented steps or acts may be combined with operator or human implemented steps or acts in order to carry out an embodiment of the invention.

As the phrase is used herein, a processor may be “configured to” perform a certain function in a variety of ways, including, for example, by having one or more general-purpose circuits perform the function by executing particular computer-executable program code embodied in computer-readable medium, and/or by having one or more application-specific circuits perform the function.

Embodiments of the present invention are described above with reference to flowcharts and/or block diagrams. It will be understood that steps of the processes described herein may be performed in orders different than those illustrated in the flowcharts. In other words, the processes represented by the blocks of a flowchart may, in some embodiments, be in performed in an order other that the order illustrated, may be combined or divided, or may be performed simultaneously. It will also be understood that the blocks of the block diagrams illustrated, in some embodiments, merely conceptual delineations between systems and one or more of the systems illustrated by a block in the block diagrams may be combined or share hardware and/or software with another one or more of the systems illustrated by a block in the block diagrams. Likewise, a device, system, apparatus, and/or the like may be made up of one or more devices, systems, apparatuses, and/or the like. For example, where a processor is illustrated or described herein, the processor may be made up of a plurality of microprocessors or other processing devices which may or may not be coupled to one another. Likewise, where a memory is illustrated or described herein, the memory may be made up of a plurality of memory devices which may or may not be coupled to one another.

While certain exemplary embodiments have been described and shown in the accompanying drawings, it is to be understood that such embodiments are merely illustrative of, and not restrictive on, the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. An apparatus for automatically detecting and classifying physical activities of a user, implemented to be worn, held by or attached to the user, comprising a computational device, an IMU (Inertial Measurement Unit) for collecting angular velocity and linear acceleration data about the user, and software for analyzing the activities of the user; wherein said computational device is configured to perform a predefined set of basic operations in response to receiving a corresponding basic instruction selected from a predefined native instruction set of codes of said software, said instruction set comprising:
a first set of machine codes selected from the native instruction set for receiving said IMU data,
a second set of machine codes selected from the native instruction set for preprocessing said IMU data and
a third set of machine codes selected from the native instruction set for determining an activity of the user according to said IMU data; wherein each of the first, second and third sets of machine code is stored in the memory.

2. The apparatus of claim 1, implemented as a mobile electronic device.

3. The apparatus of claim 2, wherein said mobile electronic device comprises a cellular telephone or a smart phone.

4. The apparatus of claim 1, implemented as a wearable.

5. The apparatus of claim 1, wherein the IMU is contained in a wearable, worn by the user, the computational device comprises a mobile electronic device and analysis is performed by said mobile electronic device upon receiving said IMU data from said wearable.

6. The apparatus of claim 1, wherein said computational device comprises a display and wherein a classified activity of the user is displayed on said display.

7. The apparatus of claim 6, wherein said software is capable of determining a length of time each activity of the user is performed, an intensity with which the activity is performed, or both, and to display said length of time, said intensity, or both, on said display.

8. The apparatus of claim 1, further comprising a GPS (global positioning system) device, for providing user location information; wherein said software analyzes said user location information as an input for determining a speed of the user in performing an activity.

9. The apparatus of claim 8, wherein said software is capable of combining previously determined GPS data and IMU data when current GPS data is not available, to determine a speed of the user from current IMU data.

10. The apparatus of claim 1, further comprising a data communication module for communicating said IMU data, said user activity information, or both, with an external device; and for receiving further analysis of said IMU data, said user activity information, or both, from said external device.

11. The apparatus of claim 10, wherein said external device comprises a server.

12. A system, comprising the apparatus of claim 1, said apparatus further comprising a data communication module for communicating said IMU data, said user activity information, or both, and further comprising a server in communication with said data communication module of said apparatus, said server further comprising a database and a classifier for classifying an activity of the user according to said IMU data, said user activity information, or both.

13. A method for analyzing physical activities of a user with the system of claim 12, comprising automatically detecting a category of physical activity of a user according to signals from the IMU.

14. The method of claim 13, further comprising automatically determining an amount of time spent in each activity.

15. The method of claim 13, further comprising automatically determining a complexity of each activity.

16. The method of claim 15, wherein said automatically determining said complexity comprises determining an entropy of said activity, wherein a higher entropy indicates a higher complexity.

17. The method of claim 13, further comprising calculating a barcode of the physical activities of the user, wherein said calculating comprises determining a physical activity type, duration, intensity and sequence of user activities.

18. The method of claim 13, further comprising receiving IMU signals from the IMU; conditioning said IMU signals to form IMU data; extracting a plurality of biomechanical parameters from said IMU data; and classifying the category of physical activity of the user according to said biomechanical parameters.

19. The method of claim 18, wherein said conditioning said IMU signals to form IMU data comprises performing a dynamic calibration so axes of the IMU are virtually aligned to functional movement axes of a movement of the user.

20. The method of claim 19, wherein said performing said dynamic calibration comprises performing an optimization that minimizes the difference between virtually-rotated-IMU signals and the functional movement axes of body segments of a body of the user.

21. The method of claim 18, wherein said extracting said biomechanical parameters comprises determining duration of movement, interpretation of intensity, calculation of velocity and IMU orientation in 3D space.

22. The method of claim 21, wherein said determining interpretation of intensity comprises determining an intensity of performance of an activity.

23. The method of claim 18, wherein said extracting said biomechanical parameters comprises determining a geometric shape of the IMU signal at each physical activity cycle and determining time series coefficients for the IMU signal.

24. The method of claim 18, wherein said classifying comprises performing a basic activity classification and an advanced activity classification, wherein said basic activity classification comprises determining an initial activity classification, and said advanced activity classification comprises determining the category of physical activity according to said initial activity classification and a temporal sequence of activity in terms of previous user activities.

25. The method of claim 24, wherein said basic activity classification further comprises applying dynamic time warping to the IMU data.

26. The method of claim 24, wherein said basic activity classification further comprises classifying the activity according to IMU signal amplitude, auto-regressive coefficients that describe each cycle of IMU data and signal form features extracted from the dynamic time warping.

27. The method of claim 13, further comprising obtaining concurrent GPS data and IMU data; correlating said GPS data and said IMU data to determine a speed of a user activity; obtaining further IMU data without GPS data; and determining said speed according to a previous correlation of said GPS data and said IMU data.
